(19) United States
(12) Patent Application Publication
(10) Pub. No.: US 2020/0003763 A1
(43) Pub. Date: Jan. 2, 2020

(54) Coated solid-phase fragments and production thereof

(71) Applicant: EUROIMMUN Medizinische Labordiagnostika AG, Luebeck (DE)

(72) Inventors: Dirk EGGERT-GOSPOS, Schuerensoehlen Schleswig-Holstein (DE); Martin Rateike, Pansdorf Schleswig-Holstein (DE); Winfried Stoecker, Gross Groenau (DE); Norbert Rottmann, Luebeck Schleswig-Holstein (DE)

(73) Assignee: EUROIMMUN Medizinische Labordiagnostika AG, Luebeck (DE)

(21) Appl. No.: 16/455,273

(22) Filed: Jun. 27, 2019

(30) Foreign Application Priority Data
Jul. 2, 2018 (EP) 18181192.8

(51) Int. Cl. G01N 33/531; G01N 33/543

(52) U.S. Cl. CPC G01N 33/531; G01N 33/54366

(57) ABSTRACT

A method is for producing biological material-coated solid-phase fragments. The method first provides a biological material-coated solid-phase support in a fragmented state and the biological material-coated solid-phase fragment adhered to a film. Then, a tensile stress is generated on the film, which is then contacted with a lifting head to pre-detach the biological material-coated solid-phase fragment from the film. The fragment is then taken off from the film. A device for producing the biological material-coated solid-phase fragment includes a tensile-stress unit, a lifting-head unit and a removal unit.

### CROSS-REFERENCE TO RELATED APPLICATIONS

The present application claims the benefit of the European Application EP18181192.8, filed on Jul. 2, 2018, which is incorporated by reference in its entirety.

### BACKGROUND OF THE INVENTION

### Field of the Invention

The present invention is directed to a method for producing biological material-coated solid-phase fragments, to a biological material-coated solid-phase fragment produced by such a method, to a support on which at least one solid-phase fragment according to the invention is applied, and to a device for producing biological material-coated solid-phase fragments that comprises a tensile-stress unit, a lifting-head unit and a removal unit.

### Discussion of the Background

Many test systems for clinical and medical diagnostics, veterinary medicine, pharmaceutical and toxicological studies, food and environmental analytical chemistry and for general-biology and biochemical analyses are based on the use of solid phase-bound bioreagents (solid-phase tests). A classic example is immunological test systems (immunoassays), without which numerous areas of modem diagnostics and analytical chemistry are impossible to imagine today. An immunoassay is a technique in which the presence of a substance is measured by means of an immunological reaction. Characteristic of every immunological reaction is an interaction between antibody and antigen. The result of said interaction is an antibody-antigen complex. The antibody or the corresponding antigen can be detected by in vitro assays. The detection of the antibody requires the addition of the relevant antigen to the test system and vice versa. Antibodies (immunoglobulins) are a particular category of proteins. Antigens can be biological or organic molecules, but also other chemical compounds. Most immunoassays require a separation of bound and free antibody and antigen (heterogeneous immunoassays or solid-phase immunoassays). Said separation is accomplished by a reaction partner (antibody or antigen) binding to a solid phase. For example, non-bound antigens can be separated from antibody-bound antigens in a simple manner when the antibody is coupled to a solid phase. If the antibody or the antigen in the form of a biomolecule (e.g. proteins, nucleic acid, polysaccharides, lectins) or as a constituent of a biological substance (tissue section, microorganism, cell) is bound to the solid phase, reference is made to a solid phase-bound bioreagent.

Solid-phase tests are generally easier-to-perform, more sensitive and simpler-to-automate than liquid-phase tests, in which the reaction partners and the reaction products are present in solution.

A particular form of diagnostic solid-phase tests is the so-called indirect immunofluorescence test (IIFT). In order to identify, for example, autoantibodies or infection antibodies in a patient sample, use is made of cells, tissue sections or purified, biochemically characterized substances as antigen substrates on a microscope slide. In a first incubation step, the antibodies to be detected from dilute patient serum bind themselves to the solid phase-bound antigens in the case of positive samples. In a second incubation step, said antibodies are visualized using fluorescently labelled anti-human antibodies. The bound antibodies and, if applicable, the specific location thereof within the substrate cell or the substrate tissue are identified under a fluorescence microscope.

Indirect immunofluorescence tests allow a high specificity, i.e. positive and negative samples yield a large difference in signal. Furthermore, what is found for each bound antibody is a typical fluorescence pattern, depending on the location of the individual antigens, and this can be utilized for identifying the autoantibodies of the patient. Furthermore, the entire antigen spectrum of the starting substrates is available, meaning that many antibodies are captured and a high hit rate can be achieved. In addition, the indirect immunofluorescence test is the method of choice when it is not possible to prepare test antigens for enzyme immunotests in a manner appropriate for the analysis.

EUROIMMUN (Lübeck, Germany) has developed a special production method and specially furnished slides for carrying out IIFTs efficiently. What are offered are slides which are furnished with multiple biological material-coated coverslips (“BIOCHIP”). BIOCHIP technology comprises an activation step in which cultured cells or tissue sections are applied to physically or chemically activated coverslips. Furthermore, frozen sections can be covalently anchored on the glass surface. The strong adherence between substrate and coverslip makes it possible to divide the coverslips containing the biological material by machine into millimetre-sized fragments (BIOCHIPs). It is thus possible to obtain 10 and more preparations of uniform quality per tissue section.

EP 0117262 B1 discloses the above-mentioned BIOCHIPs.

EP 1718948 B1 describes the above-mentioned IIFT and a corresponding BIOCHIP production method. Said production method uses a needle which pierces an adhesive film on which biological material-coated coverslip fragments are situated. The needle pierces the film such that the coverslip fragments can be removed from the film by a suction cup. However, it is not possible to produce relatively large (larger than 3.25 mm edge length) BIOCHIPs using this production method.

For different applications, for example pathological examinations of patient tissue or the simultaneous examination of multiple different tissue types, it would also be desirable to be able to produce relatively large BIOCHIPs. Therefore, there is a need for a method which allows a reproducible production of relatively large biological material-coated coverslips and for coated coverslips of this kind.

Production methods, biological material-coated solid-phase fragments, supports comprising these and devices for the production thereof, which encompass larger BIOCHIPs than known in the related art, are described below and are subject matter of the present invention.

### SUMMARY OF THE INVENTION

The inventors of the present invention have found that, surprisingly, the use of a needle in the detachment step of production methods for biological material-coated solid-phase fragments can be replaced with a pre-detachment step using a lifting head. By means of this change, it is possible to produce substantially larger solid-phase fragments/coverslip fragments.

Specifically, in the method according to the invention, a tensile stress is generated on a film coated with solid-phase fragments. Thereafter, the film is contacted with a lifting head in order to pre-detach one or more solid-phase fragments from the film.

The present invention includes the following embodiments:
1. Method for producing biological material-coated solid-phase fragments, comprising:
   providing a biological material-coated solid-phase support, the solid-phase support being in a fragmented state and the fragments adhering to a film;
   generating a tensile stress on the film coated with the solid-phase fragments,
   contacting the film with a lifting head in order to pre-detach one or more solid-phase fragments from the film; and
   taking off the one or more pre-detached solid-phase fragments from the film.
2. Method according to embodiment 1, wherein
   (a) the biological material-coated surface (2) of the solid-phase fragment comprises at least 10.25 mm², preferably at least 25 mm²;
   (b) the length of at least one edge (4, 5) of the solid-phase fragment is greater than 3.25 mm, preferably greater than 5 mm; and/or
   (c) the solid-phase fragment is a glass fragment.
3. Method according to embodiment 1 or 2, wherein the solid-phase fragment thickness (3) is between 0.05 mm and 0.3 mm, preferably between 0.1 mm and 0.2 mm.
4 Method according to any of embodiments 1 to 3, wherein the biological material is bound to the solid-phase support via a linker molecule.
5. Method according to embodiment 4, wherein the linker molecule
   (a) is a silicon-comprising compound, preferably a silyl ether; or
   (b) comprises a structure selected from the group consisting of

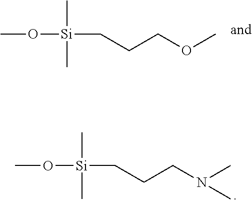

6. Method according to any of embodiments 1 to 5, wherein at least 70%, preferably at least 85%, of the surface (2) of the solid-phase fragment is coated with biological material.
7. Method according to any of embodiments 1 to 6, wherein, in the contacting step, the lifting head presses the film in the direction of the coated surface (2) of the solid-phase fragment.
8. Method according to embodiment 7, wherein the pressing due to the lifting head does not damage, tear or puncture the film.
9. Method according to any of embodiments 1 to 8, wherein the tensile stress is generated by the film being stretched by means of a frame, preferably a circular frame.
10. Method according to any of embodiments 1 to 9, wherein the take-off is achieved by means of a removal unit having a suction cup.
11. Method according to any of embodiments 1 to 10, wherein the film is an adhesive film comprising
   (a) polyvinyl chloride (PVC); and/or
   (b) acrylic adhesive.
12. Biological material-coated solid-phase fragment obtainable by the method according to any of embodiments 1 to 11.
13. Support on which at least one solid-phase fragment according to embodiment 12 is applied.
14. Device for producing biological material-coated solid-phase fragments, comprising:
   tensile-stress unit designed to generate tensile stress on a film;
   lifting-head unit designed to pre-detach solid-phase fragments from a film; and
   removal unit designed to take off solid-phase fragments from a film.
15. Device according to embodiment 14, further comprising at least one control unit.

### BRIEF DESCRIPTION OF THE DRAWINGS

FIG. 1 shows schematically the structure of a biological material-coated solid-phase fragment (1).

FIG. 2 shows schematically the structure of a device for producing biological material-coated solid-phase fragments (1), comprising a tensile-stress unit (12), a lifting-head unit (13), a removal unit (14) and a control unit (15).

FIG. 3 shows various embodiments of lifting heads.

FIG. 4 shows the positioning of a lifting head onto a film comprising solid-phase fragments (1), the pre-detachment thereof from the film and a top view of the film after the take-off of one solid-phase fragment (1). (A) Contacting of the film with a piston-shaped lifting head below one solid-phase fragment (1). (B) Pre-detachment of one solid-phase fragment (1) by a piston-shaped lifting head. (C) Pre-detachment of one solid-phase fragment (1) by a lifting-head unit (13) comprising a ballpoint pen, the tip of which serves as lifting head. (D) Film comprising a fragmented solid-phase support after the take-off of one solid-phase fragment (1).

FIG. 5 shows schematically the pre-detachment of a solid-phase fragment (1) from a film by a lifting head. (A) Adherence of a solid-phase fragment (1) to a film before contacting with a lifting head. The solid-phase fragment (1) is, with its uncoated side, in complete contact with the film. (B) Owing to the generation of the tensile stress on the film and to the contacting with a lifting head, the solid-phase fragment (1) detaches from the film in the periphery of the contact site. As a result, the solid-phase fragment (1) is, with its uncoated side, only in partial contact with the film.

FIG. 6 shows in tabular form the maximum solid-phase fragment sizes that can be produced by the method according to EP 1718948 B 1 and the method according to the invention.

### DETAILED DESCRIPTION OF THE INVENTION

In a first aspect, the present invention is therefore directed to a method for producing biological material-coated solid-phase fragments, comprising:
providing a biological material-coated solid-phase support, the solid-phase support being in a fragmented state and the fragments adhering to a film;
generating a tensile stress on the film coated with the solid-phase fragments;
contacting the film with a lifting head in order to pre-detach one or more solid-phase fragments from the film; and
taking off the one or more pre-detached solid-phase fragments from the film.

Preferred embodiments of the method according to the invention are described in what follows, wherein (a) the biological material-coated surface (2) of the solid-phase fragment comprises at least 10.25 mm², preferably at least 25 mm²; (b) the length of at least one edge (4, 5) of the solid-phase fragment is greater than 3.25 mm, preferably greater than 5 mm; and/or (c) the solid-phase fragment is a glass fragment.

In preferred embodiments, the solid-phase fragment thickness (3) is between 0.05 mm and 0.3 mm, preferably between 0.1 mm and 0.2 mm.

In further preferred embodiments, the biological material is bound to the solid-phase support via a linker molecule. The linker molecule can be (a) a silicon-comprising compound, preferably a silyl ether; or comprise (b) a structure selected from the group consisting of

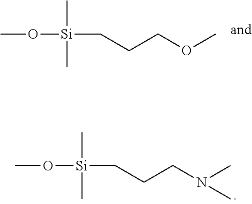

In preferred embodiments of the method according to the invention, at least 70%, preferably at least 85%, of the surface (2) of the solid-phase fragment is coated with biological material.

Furthermore, in preferred embodiments, what is comprised by the method is that, in the contacting step, the lifting head presses the film in the direction of the coated surface (2) of the solid-phase fragment. In further preferred embodiments, the film is not damaged, tom or punctured via the pressing due to the lifting head.

In further preferred embodiments, the tensile stress is generated by the film being stretched by means of a frame, preferably a circular frame.

In preferred embodiments, the take-off is achieved by means of a removal unit having a suction cup.

In further preferred embodiments of the method according to the invention, the film is an adhesive film comprising (a) polyvinyl chloride (PVC); and/or (b) acrylic adhesive.

In a second aspect, the present invention is directed to a biological material-coated solid-phase fragment obtainable by the method according to the invention.

In a third aspect, the present invention is directed to a support on which at least one solid-phase fragment according to the invention is applied.

In a fourth aspect, the present invention is directed to a device for producing biological material-coated solid-phase fragments, comprising:
tensile-stress unit designed to generate tensile stress on a film;
lifting-head unit designed to pre-detach solid-phase fragments from a film; and
removal unit designed to take off solid-phase fragments from a film.

In preferred embodiments, the device according to the invention further comprises at least one control unit.

A further aspect of the present invention is directed to a solid-phase fragment which is coated with biological material and wherein (a) the biological material-coated surface (2) of the solid-phase fragment comprises at least 10.25 mm², preferably at least 25 mm²; and/or (b) the length of at least one edge (4, 5) of the solid-phase fragment is greater than 3.25 mm, preferably greater than 5 mm. Preferably, the solid-phase fragment is a glass fragment. In preferred embodiments, the solid-phase fragment thickness (3) is between 0.05 mm and 0.3 mm, preferably between 0.1 mm and 0.2 mm. In further preferred embodiments, the biological material is bound to the solid-phase support via a linker molecule. The linker molecule can be (a) a silicon-comprising compound, preferably a silyl ether; or comprise (b) a structure selected from the group consisting of

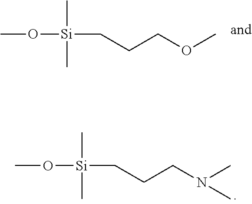

In preferred embodiments of the solid-phase fragment according to the invention, at least 70%, preferably at least 85%, of a surface (2) is coated with biological material.

The expression “biological material”, as used herein, refers to material which is isolated or derived from living organisms. Without being restricted thereto, examples of biological material encompass proteins, nucleic acids, lipids, cells, tissues, tissue sections, organs, organ sections, cell-based constructs or combinations thereof. In some aspects, biological material can refer to mammalian cells. In other aspects, biological material can refer to immortalized cell cultures, platelets, bacteria, viruses, mammalian cell membranes, liposomes, enzymes or combinations thereof. In other aspects, biological material can refer to reproductive cells, including sperm cells, spermatocytes, oocytes, egg cells, embryos, blastocysts or combinations thereof. In other aspects, biological material can refer to whole blood, serum, red blood cells, white blood cells, platelets, cerebrospinal fluid, viruses, bacteria, algae, fungi or combinations thereof. Biological material also encompasses recombinantly produced, purified or unpurified proteins and antibodies, such as a homogeneous population of monoclonal antibodies or a heterogeneous population of polyclonal antibodies.

The expression “coating”, as used in this application, is intended to refer to the application of biological material to the surface of a solid-phase fragment. Coating encompasses binding due to adhesive forces, binding due to a linker molecule, film coating and incrustation. The coating can be complete or partial, for example over 30% or more, over 40% or more, over 50% or more, over 60% or more, over 70% or more, over 75% or more, over 80% or more, over 85% or more, over 90% or more, over 95% or more, over 97% or more of the surface of the solid-phase fragment. In further preferred embodiments, the coating is preferably applied essentially over the entire surface of the solid-phase fragment.

The expression “linker molecule” or “linker”, as used interchangeably herein, refers to any molecular unit which mediates spaced bonding between two or more molecules, molecular groups and/or molecular units. Preferably, the linker molecule has a covalent bond to the solid-phase fragment and a further covalent bond to the biological material. Exemplary linkers which can be used in solid-phase fragments and methods of the present invention are polymers, for example polyethylene glycol (PEG) units, it being possible for the polymer chains to comprise additionally alkyl, alkene, alkyne, ester, ether, amide, imide, sulfo and/or phosphodiester groups. Particularly preferably, the linker molecule is a silicon (Si)-comprising compound, even further preferably a silyl ether of the base structure R₁R₂R₃Si—O—R₄, where R₁, R₂ and R₃ are non-substituted or substituted organic groups and R₄ is an alkyl or aryl group.

“Solid-phase fragment”, as used herein, refers to any solid-phase material to which a biomolecule can be affixed. Exemplary solid substrates which can be used with biological material and methods of the present disclosure encompass beads, slides, recesses and chips, which can be produced from various solid-phase materials, including glass, polymer and silicon. Particularly preferably, the solid-phase fragment is a biological material-coated glass support. The expression “solid-phase support”, as used herein, refers to a solid coated substrate as described above, but before its fragmentation. In this respect, the above-mentioned material properties of a solid-phase fragment also apply to a solid-phase support.

The expression “fragmented”, as used herein, is directed to solid-phase supports which are reduced to two or more pieces (fragments). A person skilled in the art is aware of various techniques for fragmentation, especially of coated glass supports. These encompass fracturing, cutting (by means of a cutter with blade or water jet), lasering and scratching, but are not restricted thereto. The shape of the resultant pieces can be uniform or varied. In preferred embodiments, the pieces are uniformly shaped, especially as rectangles. In yet further preferred embodiments, the pieces are squares. Preferably, the fragmented pieces have at least one surface area which is not greater than 1500 mm², not greater than 1000 mm², not greater than 750 mm², not greater than 500 mm², not greater than 400 mm², not greater than 300 mm², not greater than 200 mm² or not greater than 120 mm². In other preferred embodiments, the edge length (4, 5) of at least one side is not longer than 150 mm², not longer than 130 mm², not longer than 100 mm², not longer than 80 mm², not longer than 50 mm², not longer than 40 mm², not longer than 30 mm² or not longer than 25 mm².

The expression “film”, as used herein, refers to an adhesion film or an adhesive film. An adhesion film in the context of the present invention is a film which allows, by means of adhesive forces at its surface, an adherence to glass, preferably having a thickness between 0.001 mm and 1.5 mm. In preferred embodiments, the adhesive work, i.e. the force which must be expended to separate the adhesion film and the solid-phase fragment, is at least 0.00001 J/mm², at least 0.00001 J/mm², at least 0.00001 J/mm², at least 0.00001 J/mm², at least 0.0001 J/mm², at least 0.001 J/mm², at least 0.01 J/mm², at least 0.1 J/mm², at least 1 J/mm², at least 10 J/mm² or at least 100 J/mm².

“Adhesive film”, as used herein, is understood to mean a (plastics) film provided with an adhesive on one side or on both sides. An adhesive is natural or synthetic material which can adhere to the site of topical application. Preferably, the adhesive can adhere to glass. Further preferably, suitable adhesives are selected from polyisobutylene, acrylates, silicones, polyisoalkylenes, polyether block amide copolymers, polybutadiene, styrene-butadiene (or isoprene)-styrene block copolymer rubber, vinyl-based high-molecular-weight materials such as, for example, polyvinyl alkyl ethers, polyvinyl acetate, ethylene-vinyl acetate copolymers, partially saponified products of polyvinyl acetate, polyvinyl alcohol and polyvinylpyrrolidone, polyurethane or a combination thereof. In preferred embodiments, the adhesive is an acrylic adhesive. Acrylic adhesives encompass crosslinked and uncrosslinked acrylic copolymers, selected from polymethacrylate, such as butyl acrylate, ethylhexyl acrylate, vinyl acetate, (meth)acrylic acid, such as butyl (meth)acrylate, pentyl (meth)acrylate, hexyl (meth)acrylate, heptyl (meth)acrylate, octyl (meth)acrylate, nonyl (meth)acrylate, decyl (meth)acrylate, undecyl (meth)acrylate, dodecyl (meth)acrylate and tridecyl (meth)acrylate and copolymers of at least one of the above esters and other monomers copolymerizable therewith. Preferred acrylate polymers are commercially available under the brand name Gelva®, for example Gelva® 3011, or DuroTak® from National Starch and Chemical Company. Zutphen, the Netherlands, for example DuroTak® 202A, DuroTak® 608. DuroTak® 4201, DuroTak® 2510, DuroTak® 8710, DuroTak® 87-2353, DuroTak® 87-2353, DuroTak® 87-2353, DuroTak® 87-2353, DuroTak® 387-2051 or DuroTak® 387-2052. In further preferred embodiments, the adhesive film is the film 1009R (silicone-free blue) or the film 1020R (UV curable adhesive film (PVC)), both produced and sold by Ultron Systems (Moorpark, USA).

The film material (of adhesion film and adhesive film) can be a thin flexible film material and encompasses polymer films, such as polyvinyl chloride (PVC), polyethylene (PE), polypropylene (PP), polyethylene terephthalate (PET), polystyrene (PS), polyamide (PA), comparable polymers and combinations thereof; and metal films, such as aluminium (Al). Preferably, a polyvinyl chloride (PVC) film is used.

The expression “tensile stress” or “stress”, as used interchangeably herein, is represented by the symbol “σ” and is, on an imaginary cross-sectional area through a body, the force component Fi in the i direction based on the area A (normal n) on which it acts. It can be calculated as follows:

$${σ}_{ni} = {lim}_{Δ A \to 0} \frac{Δ {F}_{i}}{Δ A}$$

In preferred embodiments, the tensile stress on the film furnished with solid-phase fragments is at least 0.00001 N/mm², at least 0.00001 N/mm², at least 0.00001 N/mm², at least 0.00001 N/mm², at least 0.0001 N/mm², at least 0.001 N/mm², at least 0.01 N/mm², at least 0.1 N/mm², at least 1 N/mm², at least 10 N/mm² or at least 100 N/mm².

“Lifting head”, as used herein, refers to a work tool which can be contacted with a film furnished with solid-phase fragments and which is designed such that, with the aid thereof, the fragments can be removed from the film by the method according to the invention. In this respect, the lifting head has at least one side/area with which the film can be contacted. Said side/area of the lifting head can have any shape but is preferably round or oval. In other preferred embodiments, said area/side of the lifting head has a circumference or diameter of from 0.001 mm to 6 mm, from 0.05 mm to 5 mm, from 0.1 mm to 4 mm, from 0.5 mm to 3 mm, from 0.7 mm to 2 mm or from 1 mm to 1.5 mm. In further preferred embodiments, the lifting-head unit according to the invention has at least one lifting head designed as described above.

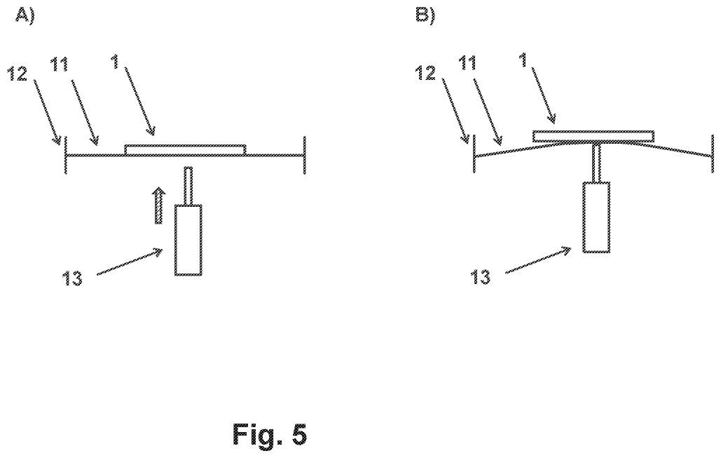

The expression “pre-detach”, as used herein, refers to the partial detachment of a solid-phase fragment from the film. In preferred embodiments, “pre-detach” refers to the detachment of at least 20%, at least 30%, at least 35%, at least 40%, at least 45%, at least 50%, at least 55%, at least 60%, at least 65%, at least 70%, at least 75%, at least 80%, at least 85%, at least 90%, at least 93% or at least 97% of a surface side of the solid-phase fragment from the film. Without being tied to any particular theory, it is suspected that the pre-detachment in the method according to the invention arises through the co-operation of the tensile stress on the film and the contacting and pressing of the lifting head in the direction of the coated surface. As a result of such a movement of the lifting head under the solid-phase fragment, the film appears to be deflected and to detach from the regions of the fragment that are not touched by the lifting head (see FIG. 5).

The expression “take off” or “remove”, as used interchangeably herein, refers to the final detachment of the solid-phase fragment from the film. This means that 100% of a previously bound surface side of the solid-phase fragment are detached from the film. After take-off, there is no direct or indirect connection between the solid-phase fragment and the film. Such a step is preferably carried out by means of a unit having a suction cup.

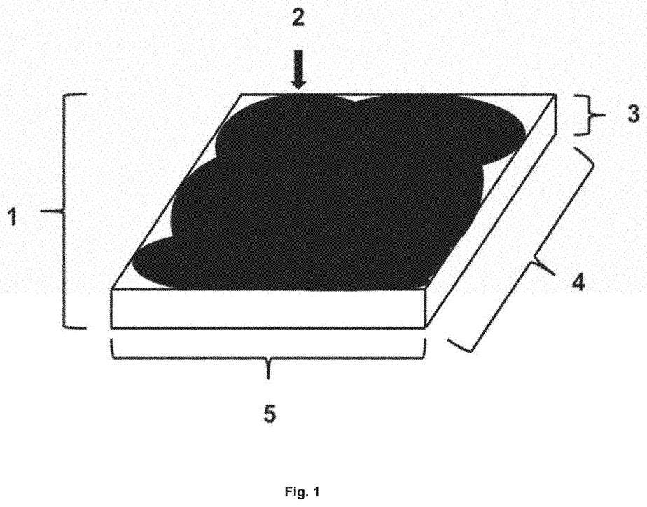

A biological material-coated surface of the solid-phase fragment is depicted in FIG. 1 under the reference sign (2). The size of the surface is determined by the edges (4) and (5). In preferred embodiments, the biological material-coated surface (2) of the solid-phase fragment comprises at least 5 mm², at least 10 mm², at least 10.25 mm², at least 15 mm², at least 20 mm², at least 25 mm², at least 30 mm², at least 40 mm², at least 45 mm² or at least 50 mm².

Edges of the biological material-coated solid-phase fragment are depicted in FIG. 1 under the reference signs (4) and (5). Preferably, the length of at least one edge (4, 5) of the solid-phase fragment is greater than 1 mm, greater than 1.5 mm, greater than 2 mm, greater than 2.5 mm, greater than 3.25 mm, greater than 4 mm, greater than 4.5 mm, greater than 5 mm, greater than 7 mm, greater than 10 mm, greater than 15 mm, greater than 20 mm or greater than 22 mm. In alternative embodiments, the two edges (4) and (5) are each greater than the aforementioned lengths.

The solid-phase fragment thickness is depicted in FIG. 1 under the reference sign 3.

Preferably, the solid-phase fragment thickness is between 0.001 mm and 1.5 mm, between 0.005 mm and 1 mm, between 0.01 mm and 0.8 mm, between 0.05 mm and 0.6 mm, between 0.06 mm and 0.5 mm, between 0.07 mm and 0.4 mm, between 0.08 mm and 0.3 mm and between 0.1 mm and 0.2 mm. For the thickness of the solid-phase support, the values are identical.

The expression “press in the direction of the coated surface”, as used herein, means that the lifting head is positioned on the film on the non-furnished side directly below a solid-phase fragment and presses said fragment upwards. Upon pressing, there is a change in, firstly, the position of the lifting head and, secondly, the positions of the film and the attached solid-phase fragments relative to one another. Firstly, this can take place by the film remaining rigidly in its position, while the lifting head is guided in a movement from bottom to top. Alternatively, the lifting head can remain rigidly in one position, while the film and the adherent solid-phase fragments are pressed by means of the lifting head. In a third alternative, the lifting head and the film can move towards one another, by both the lifting head and the film changing their positions. In preferred embodiments of the present invention, the stroke of the lifting head in the direction (or vice versa) is 0.1 mm to 30 mm, 0.3 mm to 25 mm, 0.5 mm to 20 mm, 0.8 mm to 15 mm, 1 mm to 13 mm, 3 mm to 10 mm, 4 mm to 8 mm or 5.5 mm to 6.5 mm.

“Not damage, tear or puncture”, as used herein, refers to the fact that the lifting head leaves no damage after contacting and pressing on the film. No damage in the context of the present disclosure are stretches and discolorations of the film. Damage in the context of the present disclosure is the appearance of holes or tears in the film, the appearance of protruding fibres on the film surface and the occurrence of piling, i.e. a local accumulation of film material. Therefore, the lifting head deflects the film and the solid-phase fragment after contacting only and leaves possibly stretches and discolorations of the film.

The expression “frame”, as used herein, refers to a clamping system onto which a film can be fitted. Such systems are, for example, known as clamping sets from wafer production. Useful clamping sets from inter alia Minitron Elektronik GmbH (Ingolstadt, Germany), Technovision, Inc. (Nakayama, Japan), Ultron Systems, Inc. (Moorpark, USA) and Thai-Hibex (Pathumthani, Thailand) are commercially available. In preferred embodiments, the frame described herein is part of the tensile-stress unit according to the invention.

A removal unit having a suction cup, as is suitable for use in the method according to the invention, is described by way of example in EP 1718948 B1 in paragraphs [0059] to [0061] and [0063] to [0066] and is hereby subject matter of the present disclosure by reference.

A “support”, as used herein, refers to a solid body to which the solid-phase fragments according to the invention can be attached. The support has at least one planar surface on which solid-phase fragment(s) can be applied. In preferred embodiments, the support material consists of or comprises glass, polymer or silicon. The solid-phase fragments and the support are preferably brought together by adhesive bonding, melting of at least one surface or by a physical connecting system (e.g. a tongue-and-groove connection). However, preference is given to the bonding of support and solid-phase fragment. Furthermore, at least one, at least two, at least three, at least four, at least five, at least six, at least seven, at least eight, at least nine or at least ten solid-phase fragments according to the invention are preferably applied to a support. In preferred embodiments, a support can have a size of at least 5×25 mm², at least 15×25 mm², at least 25×25 mm², at least 40×25 mm² or at least 60×25 mm². For example, a support sized at least 5×25 mm² can be furnished with altogether 5 solid-phase fragments having a size of 5×5 mm² each; 20 solid-phase fragments having a size of 2.5×2.5 mm² each; or 125 solid-phase fragments having a size of 1×1 mm² each.

A “device for producing the solid-phase fragments according to the invention”, as used herein, refers to one or more work parts comprising a tensile-stress unit, a lifting-head unit and a removal unit, as described herein. The stated units can be completely or partially separated from the other units and form a production system or all units can be connected to one another directly or indirectly.

The expression “antibody”, as used herein, refers to proteins from the class of the globulins, which are formed in vertebrates as a response to certain substances, known as antigens. Antibodies are components of the immune system. Antibodies are produced by a class of white blood cells, the B lymphocytes. They can be differentiated on the basis of different classes, namely immunoglobulin A, immunoglobulin D, immunoglobulin E, immunoglobulin G, immunoglobulin M, immunoglobulin W and immunoglobulin Y.

The expression “antigen”, as used herein, preferably refers to substances to which antibodies and certain lymphocyte receptors can specifically bind themselves. Antigens can be proteins, but also glycoproteins, carbohydrates, lipids or other substances. In the present case, the antigens are preferably proteins or post-translationally modified proteins.

In general, “at least 1”, as used herein, means 1, 2, 3, 4, 5, 6, 7, 8, 9 or more, the specification referring, where appropriate, to the type of the stated substance and not the absolute number of molecules.

Depending on the particular implementation requirements, it is possible for exemplary embodiments of the invention to realize the control unit as hardware and/or as software. The control unit mentioned here can be realized here as at least one control unit or else by multiple control units in co-operation. The implementation can be carried out using a digital storage medium, for example a floppy disc, a DVD, a Blu-Ray disc, a CD, a ROM, a PROM, an EPROM, an EEPROM or a FLASH memory, a hard drive or some other magnetic or optical memory which stores electronically readable control signals that interact or can interact with a programmable hardware component such that the particular method is carried out.

A programmable hardware component can be formed as a control unit by means of a processing unit, a central processing unit (CPU), a computer, a computer system, an application-specific integrated circuit (ASIC), an integrated circuit (IC), a system on a chip (SOC), a programmable logic element or a field-programmable gate array with a microprocessor (FPGA).

Although some aspects have been described in connection with the solid-phase fragments according to the invention, it is evident that said aspects are also a description of the corresponding (production) method and vice versa.

For the preparation for the method according to the invention, biological material-coated solid-phase supports can be produced as follows: biological substances, as mentioned above for example, are first applied to a support material, for example paper-thin (0.15 mm) glass, such as a coverslip. Generally, the biological activity of the biological substances which are used for the coating and which are referred to as substrates remains unchanged. If necessary, the biological material can be covalently bonded to the support via a linker molecule.

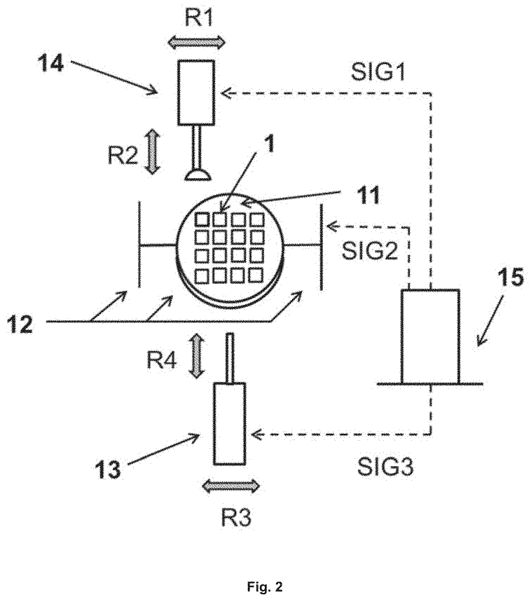

The biological substance-coated support material, for example a coverslip, is secured on a plastics film provided with an adhesive, for example in the form of a film ring, the biological substance not coming into contact with the plastics film. Said film ring can be a tensile-stress unit (12) which, in preferred embodiments, is associated with a control unit (15) and can be controlled by the control signal SIG2, for example in order to start electronically the removal process on the device according to the invention (see FIG. 2).

With the substance layer, i.e. the substrate side, downward, the coverslip is placed into a recess or stencil of a device, the uncoated coverslip edge being supported. The stencil serves for the defined orientation of the coverslip in position in relation to film and rotation angle. By means of adhesion, the coverslip is fixed to the film, the uncoated side of the coverslip meeting the film present in the film ring.

Then, the support material (coverslip) is, together with the biological substances (substrate), divided into millimetre-sized fragments (solid-phase fragments) using a diamond-equipped device (fragmenting machine or fragmenting unit) without the film being cut.

After fragmentation, the substrate-furnished coverslip on the film ring is additionally rolled between a flexible base and a pressure roller in order to fracture substrate fragments that are not yet fractured. However, the resultant substrate fragments are so close together that the edges thereof rub against one another. To prevent this, the substrate fragments can be spaced apart by concentric stretching of the film present in the film ring. For this purpose, the film clamped between the two concentric rings is stretched in all directions, for example by the larger ring being displaced by pressing into an identical ring. What are then obtained are cleanly cut fragments having precise dimensions and an exact distribution of the substrate fragments.

For the removal according to the invention of the solid-phase fragments, the film ring is placed on the film holder of a placement machine that is movable in at least one direction. The film holder is hollowed out throughout the centre.

The placement machine is loaded with analysis plates, i.e. for example printed slides, which can be supplied on a transport tray to the machine. The transport tray can have a movable side edge which fixes and thus better positions the slides.

The placement machine is equipped with a transfer unit consisting of two movable transport carriages moving on a track. Mounted on one transport carriage is a vacuum-supported suction cup which can move in at least two, preferably three directions in space, driven by a stepper motor. The suction cup is pneumatically supported and rotatable. In addition, the suction cup is part of a removal unit (14) which, in preferred embodiments, is associated with a control unit (15) and can be controlled by the control signal SIG1, for example in order to control electronically the movement of the suction cup or the entire removal unit in the directions of movement R1 and R2 (see FIG. 2).

Situated below the film ring comprising the substrate fragment-furnished film that is installed in the placement machine is a piston-type device (removal unit (13)) having a lifting head that supports the detachment of the substrate fragments from the film. The lifting head lifts slowly about 4-8 mm out of the resting position (see FIG. 5), contacts the film from below and brings about a pre-detachment. The final removal is controlled from above by the suction of the substrate fragment by the suction cup. The lifting head is part of a lifting-head unit (13) which, in preferred embodiments, is associated with a control unit (15) and can be controlled by the control signal SIG3, for example in order to control electronically the movement of the lifting head or the entire lifting-head unit (13) in the directions of movement R3 and R4 (see FIG. 2).

A second transport carriage situated on the transfer unit of the placement machine serves as a so-called “adhesive carriage”. It is equipped with a device which contains a movable adhesive needle and is connected to an adhesive reservoir via a hose. Before the application of a substrate fragment to a slide, the adhesive needle dispenses or distributes the UV adhesive, measured proportionally in volume in relation to the substrate fragment area, onto the selected placement position or surface of the slide, the adhesive needle coming into contact with the slide or into close proximity thereof. Immediately thereafter, the substrate fragment is placed onto the adhesive-wetted site of the slide. Further substrate fragments are then placed accurately on the slide in the same way. Alternatively, the adhesive can also be dispensed in a contact-free manner. Such systems are known in the related art and are, for example, sold by Nordson (Westlake, USA) in the form of “P-Jet CT Jet Valves”, high-performance jet valves for the contactless microdispensing of media of low to medium viscosity.

The placement machine (synonym “device for producing biological material-coated solid-phase fragments”) can contain units which serve for in-process control of the executed work steps and immediate error correction.

The produced supports comprising the solid-phase fragments according to the invention can then be subjected to, for example, various staining techniques (e.g. haematoxylin and eosin stain) for pathological examination. Alternatively, it is possible to carry out on the support according to the invention an IIFT test, as described by Damoiseaux et al. (Damoiseaux, J, et al. (2009) Journal of Immunological Methods, volume 348, issues 1-2, pages 67-73) for example.

FIG. 1 shows schematically the structure of a biological material-coated solid-phase fragment (1).

FIG. 2 shows schematically the structure of a device for producing biological material-coated solid-phase fragments (1), comprising a tensile-stress unit (12), a lifting-head unit (13), a removal unit (14) and a control unit (15).

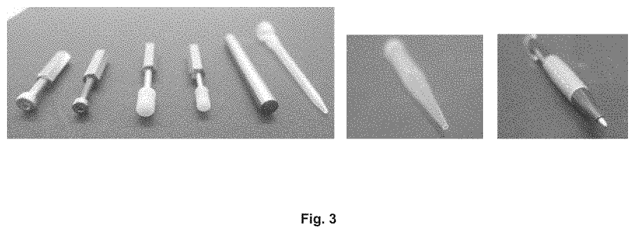

FIG. 3 shows various embodiments of lifting heads.

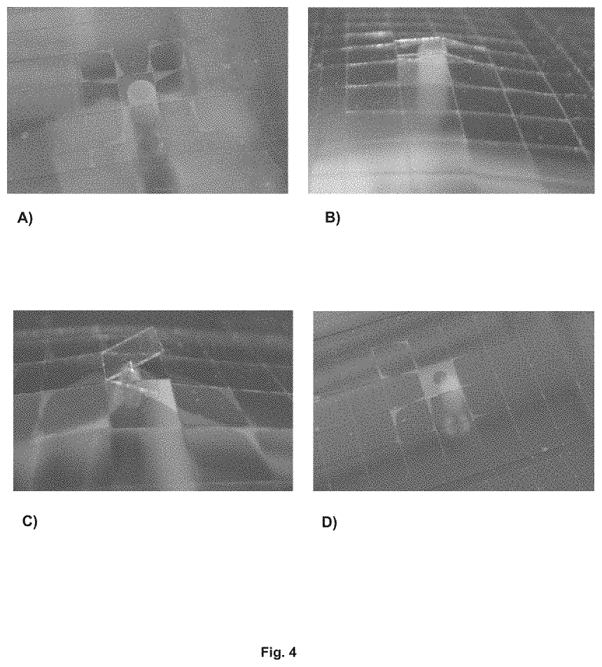

FIG. 4 shows the positioning of a lifting head onto a film comprising solid-phase fragments (1), the pre-detachment thereof from the film and a top view of the film after the take-off of one solid-phase fragment (1). (A) Contacting of the film with a piston-shaped lifting head below one solid-phase fragment (1). (B) Pre-detachment of one solid-phase fragment (1) by a piston-shaped lifting head. (C) Pre-detachment of one solid-phase fragment (1) by a lifting-head unit (13) comprising a ballpoint pen, the tip of which serves as lifting head. (D) Film comprising a fragmented solid-phase support after the take-off of one solid-phase fragment (1).

FIG. 5 shows schematically the pre-detachment of a solid-phase fragment (1) from a film by a lifting head. (A) Adherence of a solid-phase fragment (1) to a film before contacting with a lifting head. The solid-phase fragment (1) is, with its uncoated side, in complete contact with the film. (B) Owing to the generation of the tensile stress on the film and to the contacting with a lifting head, the solid-phase fragment (1) detaches from the film in the periphery of the contact site. As a result, the solid-phase fragment (1) is, with its uncoated side, only in partial contact with the film.

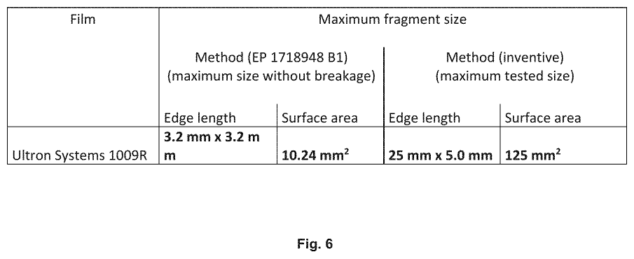

FIG. 6 shows in tabular form the maximum solid-phase fragment sizes that can be produced by the method according to EP 1718948 B 1 and the method according to the invention.

### EXAMPLES

Materials and Instruments:
Various lifting heads: ballpoint pen refills, cylinder head screws, 3D printer heads, alignment pins, pipette tips;
Vice for accommodating the lifting head;
Film, stretched in the film ring and furnished with solid-phase fragments of various formats

### Example 1: Method for Producing a Biological Material-Coated Solid-Phase Fragment with the Aid of a Pre-Detachment Step

A solid-phase support sized 76×26×0.15 mm³ was fitted onto a film (Ultron Systems dicing tape 1009R) and fragmented. The selected lifting head, for example the head of a cylinder head screw (diameter 4.4 mm), was clamped in the vice as perpendicularly as possible. The film comprising the fragmented solid-phase support of the format 4.8×4.8×0.15 mm³ was guided by hand over the lifting head such that the lifting head was centred below an individual fragment. The film was placed by hand on the lifting head slowly and as horizontally as possible (FIG. 4A). By means of uniform pressing of the film onto the lifting head, a physical pressure was exerted on the targeted fragment. Said pressure was increased until the fragment detached from the adjacent fragments of the film (FIGS. 4B and C). The detachment became apparent through a brightening of the detached film surface. After this process, the fragment only adhered to the surface where the lifting head was positioned previously. When the fragment was then detached from the film with the aid of a suction cup, the brightly discoloured film surface in the size of the lifting-head contact surface remained recognizable (FIG. 4D).

It becomes apparent that the method according to the invention does not require the film to be stretched a second time, allowing a considerable reduction of work steps. The selected fragment is lifted out of the total mass and systematically detached from the adhesive force of the film. The surrounding fragments may also be raised somewhat in line with the deflection of the film. However, the film moves back after the exertion of pressure has ended, and the fragments return to their original position.

### Example 2: Test with Various Lifting-Head Variants

In further series of experiments, the use of various lifting-head variants in the above-described method according to the invention was investigated. The lifting-head variants investigated were ballpoint pen refills, cylinder head screws, 3D printer heads, alignment pins and pipette tips (FIG. 3).

It was found that, in the case of the square format 5.0×5.0×0.15 mm³, the fragment was uniformly raised from the film above a certain diameter of the lifting head. Below this size of the lifting head, the fragment was detached from the film in a tilted manner and also had to be taken off from the film while tilted (e.g. by means of a suction cup).

The lifting heads used having a diameter of from 1.0 to 1.5 mm were capable of singularizing fragments on all tested formats from 2.5×2.5×0.15 mm³ to 5.0×20.0×0.15 mm³.

To reduce the rubbing of the lifting head on the film, especially in the case of elongated fragments, the use of, for example, ballpoint pen refill tips was found to be appropriate.

### Example 3: Production of Larger Solid-Phase Fragments Using the Method According to the Invention

The size of the maximally producible solid-phase fragments using the method according to the invention was compared with those produced using the method according to EP 1718948 B1 (FIG. 6). In both cases, the film Ultron Systems 1009R and a 0.15 mm thick solid-phase support were used.

Using the method according to EP 1718948 B1, maximum fragments of 3.2×3.2 mm² edge length and accordingly a surface area of 10.24 mm² could be produced in a reproducible manner. Using the method according to the invention, it was possible to produce fragments having an edge length of 25×5.0 mm² and a surface area of 125 mm². This corresponded to more than 12.2 times the maximum surface area of fragments produced according to the method of EP 1718948 B. The production of even larger fragments by means of the method according to the invention was not investigated.

The invention is described herein in a generic and general manner. Any of the narrower types and subgroups which fall under the generic disclosure also form part of the invention. This includes the general description of the invention with a proviso or negative restriction which removes any subject matter from a (sub)group, irrespective of whether the subject matter cut out is specifically cited here or not. Other embodiments are present herewith.

A person skilled in the art will easily recognize that the present invention is well suited to realizing the objects and to achieving the mentioned advantages and the associated goals.

Furthermore, it is readily apparent to a person skilled in the art that it is possible to make various substitutions and modifications to the invention disclosed here without deviating from the scope and spirit of the invention. The methods, uses, treatments, molecules and solid-phase fragments described herein are representative of preferred embodiments, which are exemplary and are not intended to restrict the scope of the invention. Changes therein and other uses which are included within the scope of the invention and are defined by the scope of the embodiments will occur to a person skilled in the art. The listing or discussion of a previously published document in this description should not necessarily be understood as confirmation that the document belongs to the related art or is generally known.

The invention described herein illustratively can be carried out appropriately in the absence of any element or restrictions which are not specifically disclosed here. For example, the expressions “comprising”, “including”, “containing”, etc., are read comprehensively and without limitation. The word “comprise” or variations, such as “comprises” or “comprising”, are accordingly understood to be implicit, i.e. for example, it is understood that specified numbers are included, but not excluded. In addition, the terms and expressions used herein were used as expressions of description and not of limitation, and there is no intention with such terms and expressions to limit any equivalents of the features shown and described or parts thereof. This means that various modifications within the scope of protection of the claimed invention are possible. Thus, it should be understood that, although the present invention has been specifically disclosed by means of exemplary embodiments and optional features, modifications and variations of the inventions disclosed herein can be used by experts in the field, and that such modifications and variations are to be considered to be within the scope of protection of this invention.

The content of all documents and patent documents cited herein is incorporated as a whole by reference.

### LIST OF REFERENCE SIGNS

1 Solid-phase fragment
2 Biological material-coated surface
3 Thickness
4, 5 Edge length
11 Film
12 Tensile-stress unit
13 Lifting-head unit
14 Removal unit
15 Control unit
SIG1, SIG2, SIG3 Control signal
R1, R2, R3, R4 Direction of movement

1. A method for producing a biological material-coated solid-phase fragment, said method comprising:
a) providing a biological material-coated solid-phase support, the biological material-coated solid-phase support being in a fragmented state and the biological material-coated solid-phase fragment adhering to a film;
b) generating a tensile stress on the film coated with the biological material-coated solid-phase fragment;
c) contacting the film with a lifting head in order to pre-detach the biological material-coated solid-phase fragment from the film; and
d) taking off the pre-detached biological material-coated solid-phase fragment from the film.

2. The method according to claim 1, wherein
a biological material-coated surface of the biological material-coated solid-phase fragment has an area of at least 10.25 mm²;
the length of at least one edge of the biological material-coated solid-phase fragment is greater than 3.25 mm; and/or
the solid-phase fragment is a glass fragment.

3. The method according to claim 1, wherein the biological material-coated solid-phase fragment thickness is between 0.05 mm and 0.3 mm.

4. The method according to claim 1, wherein a biological material is bound to the biological material-coated solid-phase support via a linker molecule.

5. The method according to claim 4, wherein the linker molecule
is a silicon-comprising compound; or
comprises at least one structure selected from the group consisting of

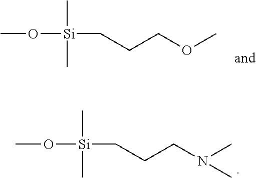

6. The method according to claim 2, wherein at least 70% of the biological material-coated surface of the solid-phase fragment is coated with the biological material.

7. The method according to claim 2, wherein, in the contacting step, the lifting head presses the film in the direction of the biological material-coated surface of the solid-phase fragment.

8. The method according to claim 7, wherein the pressing due to the lifting head does not damage, tear or puncture the film.

9. The method according to claim 1, wherein the tensile stress is generated by the film being stretched by a frame.

10. The method according to claim 1, wherein the taking off step is achieved by a removal unit having a suction cup.

11. The method according to claim 1, wherein the film is an adhesive film comprising
polyvinyl chloride (PVC); and/or
acrylic adhesive.

12. The biological material-coated solid-phase fragment obtainable by the method according to claim 1.

13. A support on which at least one of the biological material-coated solid-phase fragment according to claim 12 is applied.

14. A device for producing a biological material-coated solid-phase fragment, comprising:
a) a tensile-stress unit designed to generate tensile stress on a film;
b) a lifting-head unit designed to pre-detach the biological material-coated solid-phase fragment from the film; and
c) a removal unit designed to take off the biological material-coated solid-phase fragment from the film.

15. The device according to claim 14, further comprising at least one control unit.

16. The method according to claim 2, wherein
the biological material-coated surface of the biological material-coated solid-phase fragment has an area of at least 25 mm²; and
the length of the at least one edge of the biological material-coated solid-phase fragment is greater than 5 mm.

17. The method according to claim 3, wherein the biological material-coated solid-phase fragment thickness is between 0.1 mm and 0.2 mm.

18. The method according to claim 5, wherein the silicon-comprising compound is a silyl ether.

19. The method according to claim 6, wherein at least 85% of the biological material-coated surface of the solid-phase fragment is coated with the biological material.

20. The method according to claim 9, wherein the frame is circular.
